(19) United States
(12) Patent Application Publication
(10) Pub. No.: US 2013/0029362 A1
(43) Pub. Date: Jan. 31, 2013

(54) MARKERS AND ASSAYS FOR DETECTION OF NEUROTOXICITY

(71) Applicants: Andreas Jeromin, Alachua, FL (US); Oleana Glushakova, Alachua, FL (US); Kevin Ka-wang Wang, Gainesville, FL (US); Zhiqun Zhang, Alachua, FL (US); Ronald L. Hayes, Alachua, FL (US)

(72) Inventors: Andreas Jeromin, Alachua, FL (US); Oleana Glushakova, Alachua, FL (US); Kevin Ka-wang Wang, Gainesville, FL (US); Zhiqun Zhang, Alachua, FL (US); Ronald L. Hayes, Alachua, FL (US)

(21) Appl. No.: 13/638,611

(22) PCT Filed: Apr. 1, 2011
(86) PCT No.: PCT/US11/31029

§ 371 Date: Sep. 30, 2012

Related U.S. Application Data
(60) Provisional application No. 61/465,870, filed on Mar. 25, 2011.
(60) Provisional application No. 61/376,967, filed on Aug. 25, 2010.
(60) Provisional application No. 61/320,122, filed on Apr. 1, 2010.

(51) Int. Cl. G01N 33/53; G01N 21/64

(52) U.S. Cl. 435/7.92; 436/501

(57) ABSTRACT

A process and assay for diagnosing neurotoxicity in a subject is provided. The extent of a neurotoxic insult to a subject is assessed through the measurement of one or more biomarkers in a biological fluid, such as CSF or serum. Other uses and advantages afforded include pre-market drug discovery, monitoring, drug neurotoxicity screening and post market assessment of safety and monitoring for drug of known potential neurotoxicity.

### CROSS REFERENCE TO RELATED APPLICATIONS

This application claims priority to U.S. Provisional Application No. 61/320,122 filed Apr. 1, 2010, U.S. Provisional Application No. 61/376,967 filed Aug. 25, 2010 and Provisional Application No. 61/______ filed on Mar. 23, 2011 entitled Markers and Assays for Detection of Neurotoxicity, the entire contents of these priority documents are incorporated herein by reference.

### FIELD OF THE INVENTION

The present invention is related to the identification and use of markers of neurotoxicity. Inventive markers include proteins; or protein fragments; auto-antibodies; DNA; RNA; or miRNA. Inventive biomarkers may play a role in central nervous system function and therapy.

### BACKGROUND OF THE INVENTION

Neurodegeneration and Neurotoxicity are a safety risk associated with some compounds that are pharmacologically active in the central nervous system (CNS). However, monitoring neurodegeneration is difficult in both pre-clinical and clinical drug development. A quantitative cerebral spinal fluid (CSF) and blood-based protein biomarker of neurodegeneration and neurotoxicity could improve non-clinical safety assessments and our ability to monitor patient safety in clinical studies compared to reliance histological assessment of brain pathology in pre-clinical models in patients.

Traumatic, ischemic, and neurotoxic chemical insult, along with generic disorders, all present the prospect of brain or other neurological damage. While the diagnosis of severe forms of each of these causes is straightforward through clinical response testing and computed tomography (CT) and magnetic resonance imaging (MRI) testing, these diagnostics have their limitations in that spectroscopic imaging is both costly and time consuming while clinical response testing of incapacitated individuals is of limited value and often precludes a nuanced diagnosis. Additionally, owing to the limitations of existing diagnostics, situations under which a subject experiences a stress to their neurological condition such that the subject often is unaware that damage has occurred or seek treatment as the subtle symptoms often quickly resolve. The lack of treatment of these mild to moderate challenges to neurologic condition of a subject can have a cumulative effect or subsequently result in a severe brain damage event which in either case has a poor clinical prognosis.

In order to overcome the limitations associated with imaging and subjective clinical response diagnosis of neurological condition, there is increasing attention on the use of biomarkers as internal indicators of change as to molecular or cellular level health condition of a subject. As detection of biomarkers uses a sample obtained from a subject and detects the biomarkers in that sample, typically cerebrospinal fluid, blood, or plasma, biomarker detection holds the prospect of inexpensive, rapid, and objective measurement of neurological condition. With the attainment of rapid and objective indicators of neurological condition allows one to determine severity of a non-normal brain condition on a scale and with a degree of objectivity to thereby, predict outcome, guide therapy of the condition, as well as monitor subject responsiveness and recovery. Additionally, such information as obtained from numerous subjects allows one to gain a degree of insight into the mechanism of brain injury.

Of equal importance is the measurement or identification of toxicity associated with chemical insults or candidate therapeutics. A significant percentage of drug candidates are pulled from clinical trials due to unforeseen toxicity. The number of chemical compounds that do not survive the preclinical stages due to toxicity issues is even greater. There remains a disconnect between the currently employed toxicity screens and prediction of toxicity in humans. This is particularly true with respect to neurotoxicity. Neurotoxicity is both difficult to measure and often unrelated to the known mechanism of the drug candidate. Better measurements of neurotoxicity are needed to allow early detection of potential unwanted side effects prior to entering clinical trials.

Exposure to chemical or biological agents such as during drug or other therapeutic candidate screening remains difficult to access. These studies commonly analyze neuronal cell death. However, there is a need for compositions and methods useful for detecting alterations in neuronal cell function, structure, organization or other less severe outcome from challenge. Biomarkers of central nervous system (CNS) neurotoxic insult such as those provided by this invention could impart scientists with quantifiable neurochemical markers to help determine not only the severity and cellular pathology of neurotoxicity, but also provide a surrogate marker of therapeutic interventions.

Thus, a need exists for a sensitive and specific biochemical marker(s) of neurotoxicity that may also improve diagnostic ability and patient management, and facilitate therapeutic evaluation.

Glutamate induced excitotoxicity has been established as a model system for neurodegeneration well as many other neurological disorders, as one of the major contributory factors in triggering axonal and dendritic degeneration, which results in neuronal injury. The neuropathological consequences of TBI are mediated through sustained glutamate induced increase in cytosolic Ca2+ leading to activation of proteases such as calpain and caspase fragmentation of certain proteins and selected release of brain proteins/fragments into biofluids (e.g. CSF, blood, plasma, serum), which are considered as biomarkers of brain injury. Kainic acid (KA) is a known excitotoxin that activates subclass of ionotropic glutamate receptor, and it is neurotoxic even when administrated subcutaneously (sq.).

Thus, a need also exists for a rat KA excitotoxicity model to examine the distribution, relationship and localization of several biomarkers of TBI: ubiquitin C-terminal hydrolase 1 (UCHL 1), glial fibrillary acidic protein (GFAP), αll-spectrin break down products (SBDP150, SBDP145 and SBDP120). Degeneration of neurons in particular in a brain region known as the hippocampus leads to an imbalance of excitation and inhibition, which manifests itself as seizures. The biomarkers are thereby also of important for the detection and prediction of seizures in animal models and patients. In addition, seizures have been described as one of the long-term complications in TBI.

In addition, chemotherapy-induced cognitive decline for example in breast cancer has been recognized one of the severe side-effects of this therapeutic regiment. Cancer is the leading cause of cancer related deaths in women worldwide and in the USA in particular. Chemotherapy remains the chief and often only available curative therapy for breast cancer since the discovery of chemotherapeutic agents in the 1950s and 1960s. However, the use of this effective therapy is often limited by its adverse side effects. Neurotoxicity with the consequent neuropathy is the generally recognized main side effect for the various systemic agents of the most widely used and effective breast cancer treatments including taxanes (i.e., paclitaxel and docetaxel) and the platinum compounds (i.e., cisplatin, oxaliplatin, carboplatin). Neurotoxicity generated by paclitaxel and cisplatin can limit treatment dosage or cause their discontinuation and thereby diminishing their usefulness and effectiveness. In addition, neuropathy can significantly impair a patient's quality of life. Although PNS neurotoxicity is a common and well-studied adverse effect (Murillo et al., 2008), several reports indicate these agents can also be neurotoxic to the CNS as well (Perry and Warner, 1996). Therefore, the discovery of novel biomarkers for early detection of PNS and CNS neuropathies due to breast cancer chemotherapy lead to a more informed selection of the appropriate treatment strategies to minimize the severity or risk of long term neuropathy as an adverse consequence.

Thus a need exists for a process of using biomarkers to detect neurotoxicity in drug discovery or as an adjunct to therapeutic administration. There further exists a need for a process to monitor neurotoxicity and neuropathy in the CNS and PNS induced by chemotherapeutics for an early detection of neurotoxicity control dosing to limit neurotoxicity especially for breast cancer chemotherapeutics and allow for an early therapeutic intervention to minimize the neurotoxic side-effects of chemotherapy.

### BRIEF DESCRIPTION OF THE DRAWINGS

FIG. 1 illustrates Neurotoxicity biomarker elevation in biofluid compartment following neurotoxic response to Methamphetamine or cisplatin.

FIG. 2 depicts the time dependent effect of kainic acid (KA) (9 mg/Kg) administration on the levels of GFAP and UCH-L1 in the rat CSF.

FIG. 3 depicts the time dependent effect of KA (9 mg/Kg) administration on spectrin breakdown products in the rat CSF.

FIG. 4A depicts a Western blot for SBDP145 and SBDP150, FIG. 4B. and FIG. 4C are bar graphs depicting the time dependent effect of KA (9 mg/Kg) administration on spectrin breakdown products in the rat hippocampus derived from the Western blot of SBDP145 and SBDP150 spectrin breakdown products.

FIG. 5 are histology images showing increased level of αII-spectrin breakdown products in hippocampus of KA treated rats.

FIG. 6 are histology images showing increased level of GFAP expression in hippocampus of KA treated rats.

FIG. 7 are histology images showing increased level of activated Caspase-3 in hippocampus of KA treated rats.

FIG. 8 depicts a Western blot showing α-internexin as biomarker (α-internexin-BDP) released into human TBI CSF samples.

FIG. 9 depicts a Western blot for α-internexin where α-internexin (54 kDa) levels do not change in adult versus embryonic day 18 rat brain.

FIG. 10 depicts a Western blot for Nestin protein levels in adult versus embryonic day 18 rat brain.

FIG. 11 depicts a comparison of Western Blot for α-internexin as biomarker (α-internexin-BDP) released into human TBI CSF samples.

### DETAILED DESCRIPTION OF THE PREFERRED EMBODIMENTS

Historically, the brain is considered immunologically privileged due to the lack of a lymphatic system and protection of blood-brain barrier (BBB). Injury to the brain either from chemical, percussive, or other injury, however, often causes damage to the brain tissue or the BBB leading to the release of antigens such as peptides, degraded protein fragments, proteins, DNA and RNA (including miRNA) into the cerebrospinal fluid (CSF) or the blood stream, with subsequent increased formation of the autoantibodies against them. The present invention has utility in the detection of normal or abnormal neurological condition either in vivo or in vitro caused by chemical toxicity, physical trauma, disease, or infection through detection of cellular material released. Specifically, the invention has utility for screening assays or diagnosis of neurotoxicity due to chemical or other insult.

The subject invention also has utility as a means of detecting neurological trauma or condition that is predictive or indicative of future disease or injury. Illustratively, the subject invention has utility as a safety or efficacy screening protocol in vivo or in vitro for drug development. Drug development is not limited to drugs directed to neurological conditions. In a preferred embodiment the inventive biomarkers have utility to detect expected or unexpected neurological side effects in in vitro animal studies as a means of selecting a lead compound for analyses or as a means of assessing safety of a previously identified drug candidate. It is appreciated that a drug whether an approved commercial therapeutic (e.g. a chemotherapeutic) or a drug candidate is readily administered to a subject with monitoring of at least one biomarker as detailed herein to check for the induction of a neurological condition as a basis for determining maximal dosing levels to which a subject can be exposed before condition onset. It is further appreciated that different individuals will have specific drug tolerance thresholds and the present invention thus represents an adjunct monitoring process associated with administration of compounds having the potential for neurotoxicity.

This invention provides identification of biomarkers such as UCH-L1, GFAP, MAP-2, S100β, and spectrin-breakdown products, such as SBDP145, SBDP150, SBDP150i and SBDP120 in various tissues of a subject and the correlation of their concentration with neurodegenerative diseases and tauopathies. The invention also includes methods for the use of biomarkers in the diagnosis of neurological condition. One such approach is the detection of one or a multiplexed panel of biomarkers in brain extract after their separation by chromatography (e.g. electrophoresis and Western blotting) and in brain tissue sections (immunohistochemistry). An additional application is the detection and quantification of one or a multiplexed panel of these biomarkers in biofluids (such as CSF, serum, plasma and urine) in patients for diagnostic purposes and to monitor therapeutic interventions.

The present invention also provides a method for safety assessment drug discovery, monitoring, drug neurotoxicity screening and post market assessment of safety and monitoring for drug of known potential neurotoxicity. For example monitoring of response to cancer drugs to prevent or minimize adverse effects of these drugs such as post-chemotherapy cognitive impairment (PCCI, known as chemo brain) and chemo-induced peripheral neuropathy (CIPN)).

As a result of the Kainic Acid's properties being a potent central nervous system stimulant, and induction of seizures in experimental animals, the present invention also provides for a health assessment of patients with epilepsy, status epilepticus or single seizures including provoked seizures, post market assessment of drugs causing seizures due to overdose such as antipsychotics, and assessment of impairments causing by illegal drugs or alcohol as well as withdrawal thereof. Long term exposure to neurotoxins, such as Kainic Acid, is known to contribute to neurodegenerative disorders such as Alzheimer's Disease, thus the present invention also provides a metric to diagnose neurodegenerative disorders caused by long-term exposure to neurotoxins.

As used herein an injury is an alteration in cellular or molecular integrity, activity, level, robustness, state, or other alteration that is traceable to an event or insult. Injury illustratively includes a physical, mechanical, structural, chemical, biological, functional, infectious, or other modulator of cellular or molecular characteristics. An event is illustratively, a chemical or biological insult such as exposure to a chemical or biological agent. An event is optionally an infection by an infectious agent. A person of skill in the art recognizes numerous equivalent insults that are encompassed by the terms injury or event. It is appreciated that such an agent represents a therapeutic administered to treat a condition, yet has neurotoxicity at certain doses or in certain subjects.

The term “biomarker” as used herein represents antibodies, DNA, RNA, miRNA, fragments of RNA, fragments of DNA, peptides, proteins, lipids, or other biological material whose presence, absence, level or activity is correlative of or predictive of neurological toxicity, damage, or disease. Optionally, a biomarker is a protein. Alternatively or in addition, an inventive biomarker is a portion of or the full length version of oligonucleotides or peptides that encode or are: αII-spectrin; a spectrin breakdown product (SBDP) illustratively SBDP150, SBDP150i, SBDP145, and SBDP120; GFAP; neuron specific enolase (NSE); neurofilament protein light chain (NFp), α-internexin; nestin, ubiquitin C-terminal hydrolase L1 (UCH-L1); Neuronal Nuclei protein (NeuN); 2′, 3′-cyclic nucleotide 3′-phosphodiesterase (CNPase); Soluble Intercellular Adhesion Molecule-1 (sICAM-1); inducible nitric oxide synthase (iNOS); or other marker listed in Table 1. Optionally, an inventive biomarker recognizes, encodes or is UCH-L1 or SBDP150 or SBDP145. Neuron specific enolase (NSE) is found primarily in neurons. GFAP is found only in Schwann cells. CNPase is found in the myelin of the central nervous system.

TABLE 1

| | | |
|---|---|---|
| UCH-L1 | Glycogen phosphorylase, (BB-form)GP- | Precerebellin |
| | BB | |
| MBP isoforms | CRMP-2 | Cortexin |
| SBDP150 (calpain) | NP25, NP22; Transgelin-3 | EMAP-II |
| SBDP120 (caspase) | SBDP150i (caspase) | Calcineurin-BDP |
| MBP-fragment (10/8K) | CaMPK-IIα | MAP2 |
| SBDP45 | MOG | N-Cadherin |
| Synaptophysin | PLP | N-CAM |
| βIII-Tubulin | PTPase (CD45) | Synaptobrevin |
| Tau-BDP-35K (calpain) | Nesprin-BDP | MAP1A (MAP1) |
| NF-L-BDP1 | OX-42 | MAP1B (MAP5) |
| NF-M-BDP1 | OX-8 | Prion-protein |
| NF-H-BDP1 | OX-6 | PEP19; PCP4 |
| Synaptotagmin | CaMPKIV | Synaptoagmin-BDP1 |
| PSD93-BDP1 | ynamin | BDNF |
| AMPA-R-BDP1 | Clathrin HC | Nestin |
| NMDA-R-BDP | SNAP25 | IL-6 |
| SBDP150i (caspase) | Profilin (BDP?) | IL-10 |
| MAP2-BDP1 (calpain) | Cofilin (BDP?) | αII-spectrin SBDP 150 + 145 |
| MAP2-BDP2 (caspase) | APP-BDP (Calpain) | NG2; Phosphacan, neruocan; versican |
| alpha-synuclein | NSF | Ach Receptor fragment (Nicotinic, |
| | | Muscarinic) |
| Synapsin 1 | IL-6 | I-CAM |
| Synapsin 2-BDP | MMP-9 | V-CAM |
| NeuN | S100β | AL-CAM |
| GFAP | Neuroglobin | CNPase |
| p24; VMP | UCH-L1 autoantibody | Neurofascins |
| PSD95 | Tau-BDP-35K (calpain) | Neroserpin |
| α1,2-Tubulin | Tau-BDP-45K (caspase) | EAAT(1 and 2) |
| β1,2-Tubulin | Huntingtin-BDP-1 (calpain) | Nestin |
| Stathmin-2,3,4 (Dendritic) | Huntingtin-BDP-2 (caspase) | Synaptopodin |
| Striatin-BDP1 | Prion-protein BDP | |
| Snaptojanin-1,2-BDP1 | MBP (N-term half) | |
| betaII-Spectrin | | β-synuclein |
| betaII-Spectrin-BDP110 (calpain) | Calbindin-9K | Resistin |
| betaII-Spectrin-BDP85 (caspase) | Tau-Total | Neuropilins |
| Cannabinoid-receptor1(CB1) | NSE | Orexin |
| Cannabinoid-receptor2(CB2) | CRMP-1 | Fracktalkine |
| MBP isoforms 14K + 17 | CRMP-3 | β-NGF |
| Neurocalcin-delta (Glia) | CRMP-4 | L-selectin |
| Iba1 (Microglia) | CRMP-5 | iNOS |
| Peripherin (PNS) | | |
| LC3 | Crerbellin 3 | DAT |

Non-erythroid alpha-II spectrin is a cytoskeletal protein that is cleaved by the protease calpain when this enzyme is activated by an influx of calcium into injured cells. αII-spectrin breakdown products of 150, 145, and 120 kDa (SBDP-150, -145, and -120, respectively) increase in concentration with increased magnitude of traumatic brain injury in rats and humans. Similar influxes of cellular calcium that occur during compound-induced neurotoxicity cause sufficient activation of calpain to generate SBDP's detectable in CSF, thus SBDP's are sensitive biomarkers of compound-induced neurodegeneration. The neurodegeneration is characterized by histopathology and measured SBDP-145 concentrations in CSF consistently observed >3-fold increases in SBDP-145 concentration in rats with minimal-slight lesions, and 20-150-fold increases with more severe lesions.

The present invention is optionally described with respect to UCH-L1, SBDP150 or SBDP 145. It is appreciated that these biomarkers are presented for illustrative purposes only and are not meant to imply expressly or otherwise that the scope of the present invention is limited to UCH-L1 or SBDP 145. It is appreciated that the inventive methods and compositions for detecting neurotoxicity in a subject are equally applicable to other biomarkers illustratively including those listed in Table 1. One of skill of art should appreciate that ELISA test panels the inventive biomarkers are readily performed in sequence or in parallel using antibodies reactive with the aforementioned biomarkers. Similarly, a polynucleic acid relating to one of the aforementioned biomarkers is readily replicated through conventional techniques such as PCR to detect neurotoxicity.

The use of kainic acid also provides the following major developments: 1) Development of biomarkers (single/panels) for the assessment of drug safety in pre-clinical and clinical studies (kainate as example, other compounds are possible), since current approaches rely on the histological assessment of brain sections in pre-clinical models and do not easily translate into clinical studies; 2) Development of a panel of protein-based biomarker for the detection of neurodegeneration (specifically for kainate) in-vivo in biofluids such as CSF, plasma and serum. Current data are only for CSF; and 3) Development of a panel of protein-based biomarkers for the detection of seizures, specifically based on the kainate-induced model of seizures.

Other compounds which may be used in an alternate embodiment include Chloropropionic acid (Sigma cat #306797), Bromethalin (Bell Labs, CAS #63333-35-7) and Pentylenetetrazole (PTZ) (Sigma cat # P6500), paxlitaxel, and organo-platinum compounds such as the prototypical cisplatin.

As used herein the terms “neurotoxicity” and “neurotoxic insult” are directed to reversible or irreversible changes in neuronal cells or other cells (e.g. glia cells, oligodendrocytes or Schwann cells, microglia cells) in either the central nervous system (CNS) or the peripheral nervous system (PNS) that may or may not lead to cell death. Neurotoxicity includes structural changes such as alterations in the structure or organization in branches of dendritic trees, or molecular or macromolecular reorganization such as changes related to cell metabolism, neurotransmitter pathways, and the like. Neurotoxicity is illustratively due to: radiation damage; mitochondrial poisoning; blockage of protein synthesis such as by puromycin or cycloheximide; blockage, acceleration or onset of abnormal protein synthesis including gene transcription and mRNA translation such as by actinomycin-D or ion channel blockers; or alterations in protein degradation events leading to increased buildup of one or more proteins within, secreted by, or otherwise associated with a neuronal cell. Neurotoxicity also include chemical—(such as cancer drugs or chemotherapy) induced peripheral neuropathy or chemotherapy induced changes in brain structure or functions (also “chemobrain”).

An inventive biomarker is illustratively a peptide or a protein. Detection of the presence or absence or protein, or increases or decreases in protein levels correlates with the level of neurotoxicity. As used herein, “peptide” means peptides of any length and includes proteins. The terms “polypeptide” and “oligopeptide” are used herein without any particular intended size limitation, unless a particular size is otherwise stated.

Illustrative examples of protein biomarkers include UCH-L1 and alpha-II-spectrin for alpha-II spectrin breakdown products (SBDP). Alpha-II-spectrin is primarily enriched in brain and is localized in neurons rather than glia. Furthermore, alpha-II-spectrin appears to be localized in axons (Czogalla and Sikorski, 2005; Riederer et al., 1986). Alpha-II-spectrin is cleaved by two cysteine proteases: calpain and caspase. Calpain, which exists in a quiescent state in the resting cell, is induced to a hyperactive state in response to significant elevations in intracellular calcium, and accompanies TBI (Fineman et al., 1993). This enzyme cleaves alpha-II-spectrin into 150 and 145 kDa fragments. Calpain proteolysis is primarily associated with necrotic oncosis (Kampfl et al., 1997; Liu et al., 2004; Wang, 2002). Caspase, the activation of which is associated with apoptotic cell death, cleaves spectrin into distinct 150 and 120 kDa fragments (Pike et al., 2001; Wang, 2000). This differential cleavage permits not only an indication of CNS-specific hyperactivation of spectrin cleavage enzymes in response to neurotoxic insult, but also an assessment of the relative significance of necrosis and/or apoptosis as contributory factors in the injury pathology.

An inventive biomarker is optionally a polynucleic acid such as an oligonucleotide. An oligonucleotide is a DNA or RNA molecule. Examples of RNA molecules illustratively include are mRNA and miRNA molecules. RNA molecules were historically believed to have short half-lives in plasma. More recently, studies indicated that RNA molecules may be protected in plasma by protein or lipid vesicles. As such, RNA molecules released following or neurotoxic insult, for example, can be detected in cells, tissue, blood, plasma, serum, CSF, or other biological material and be associated with the presence of injury in the inventive method. Numerous methods are known in the art for isolating RNA from a biological sample. Illustratively, the methods described by El-Heihaway, T, et al., Clinical Chem., 2004; 50(3);564-573, the contents of which are incorporated herein by reference, are operable in the present invention.

In some embodiments UCH-L1 RNA is detected. Human UCH-L1 RNA or cDNA derived therefrom is of known sequence and can be found in the NCBI database at accession number NM_{—}004181. A person of ordinary skill in the art knows that other TBI relevant RNA sequences can similarly be found in the NCBI database such as those encoding proteins listed in Table 1. As a further example, the mRNA sequence for GFAP is found at accession number NM_{—}001131019.1 and NM_{—}002055.3 for two isoforms of GFAP. The complete cDNA and protein sequence for human alpha spectrin is found at accession number M61877 J05244. The contents of each file at each accession number are incorporated herein by reference.

Primer and probe designs are within the level of skill in the art. Any suitable primer and probe as well as labels thereon are operable for the detection of mRNA biomarkers in the subject invention. Illustratively, primer and probe design can be performed using automated programs available from commercial sources. Alternatively, numerous commercial suppliers provide primer and probe design services including Applied Biosystems (Foster City, Calif.).

An inventive method for RNA illustratively includes obtaining a biological sample from a subject that may be suspected of having a neurological condition; obtaining RNA from said sample; analyzing the RNA for the presence of an RNA biomarker; comparing the level of RNA biomarker detected with the level of RNA biomarker from a subject without a neurological condition; and diagnosing the presence or absence of a neurological condition in the suspect subject.

Optionally, the inventive method involves analyzing the biological sample for the presence of a plurality of biomarkers. A plurality can be any number greater than one. Optionally, two biomarkers are analyzed. Optionally, the biomarkers are UCH-L1 and SBDP 145. More biomarkers may be simultaneously or sequentially assayed for in the inventive method illustratively including three, four, five, six, seven, eight, nine, 10, 20, 50, 100, 1000, or any number between or greater.

Optional methods for the detection and quantitation of biomarkers include real-time PCR (RT-PCR). RT-PCR allows for the simultaneous amplification and quantitation of a plurality of biomarkers simultaneously. Alternatively, mass spectroscopy such as electrospray ionization mass spectroscopy coupled with time of flight detection and high performance liquid chromatography are similarly operable. It is appreciated that other methods are similarly operable for detection as will be appreciated by one of ordinary skill in the art.

Numerous miRNA molecules are operable as biomarkers in the subject invention. The term “miRNA” is used according to its ordinary and plain meaning and refers to a microRNA molecule found in eukaryotes that is involved in RNA-based gene regulation. Examples include miRNA molecules that regulate the expression of one or more proteins listed in Table 1. Several miRNA molecules have been identified and are operable as biomarkers in the inventive methods. Illustratively, miRNA molecules described by Redell, J B, et al., J. Neurosci. Res., 2009; 87:1435-48; Lei, P., et al., Brain Res., doi:10.1016/j.brainres.2009.05.074; Lu, N, et al., Exp. Neurology, 2009; doi:10.1016/j.expneurol.2009.06.015; and Jeyaseelan, K, et al., Stroke, 2008; 39:959-966, the contents of each are incorporated herein by reference for the miRNAs defined therein, but also specifically for methods of isolation and quantitation of miRNA described in each reference. These methods, or modifications thereof, that are recognized by one of ordinary skill in the art are used in the present inventive method.

An optional method includes detection of auto-antibodies directed to antigens released from a site of neurological insult such as chemical insult illustratively by candidate drugs, disease, injury or other abnormality. Without being limited to a particular theory, a neurotoxic insult causes cellular damage that releases intracellular or cell membrane contents into the CSF or bloodstream or other biofluids such as urine, saliva, sweat, tears). The levels of many of these proteins such as those listed in Table 1 are not normally present in biological fluids other than the cytoplasm or cell membrane of neuronal tissue such as brain tissue or if they are present their levels are altered by neurotoxic insult. The presence of these antigens often leads to the production of autoantibodies to these antigens within a subject. Detection of an autoantibody as a biomarker is a preferred method of diagnosing the presence of an abnormal neurological condition in a subject.

U.S. Pat. No. 6,010,854 describes methods of producing screening antigens and methods of screening for autoantibodies to neuronal glutamate receptors. These methods are equally applicable to the subject invention. As such, U.S. Pat. No. 6,010,854 is incorporated herein by reference for its teaching of methods of producing screening antigens that are operable for screening for autoantibodies. U.S. Pat. No. 6,010,854 is similarly incorporated herein by reference for its teaching of methods of detecting autoantibodies. It is appreciated that other methods of detecting antibodies illustratively including ELISA, Western blotting, mass spectroscopy, chromatography, staining, and others known in the art are similarly operable.

Alternatively, full length protein such as any protein listed in Table 1 is operable as a screening antigen for autoantibodies. For example, UCH-L1 is antigenic and produces autoantibodies in a subject. The sequence for human UCH-L1 protein is found at NCBI accession number NP_{—}004172.2. Similarly, the sequence for human GFAP is found at NCBI accession number NP_{—}002046.1. A sequence for alpha spectrin including the sequence for SBDP 145 is listed at accession number M61877 J05244. Other optional antigens illustratively include, alpha-spectrin, SBDP 145, MAP, Tau, Neurofascin, CRMP-2, MAP2 crude sample, and human brain lysate.

Any suitable method of producing peptides and proteins of Table 1 are operable herein. Illustratively, cloning and protein expression systems used with or without purification tags are useful. Optional methods for production of immunogenic peptides includes synthetic peptide synthesis by methods known in the art. Either method is operable for the production of antigens operable for screening biological samples for the presence of autoantibodies.

It is appreciated that the patterns of biomarkers such as peptide, RNA, miRNA, DNA, and autoantibodies is operable to locate the site and severity of neuronal abnormality. Illustratively, damage to the brain reveals a different pattern of a plurality of biomarkers than does damage to other regions of the central nervous system. Also, damage to the hippocampus will produce a different pattern of biomarkers than damage to the frontal lobe. As such, localization of injury is achieved by comparative detection of a plurality of biomarkers. For example, miRNA levels within cells are altered in specific patterns in response to brain injury. (See Redell, J, et al., J. Neurosci. Res., 2009; 87:1435-1448.)

The present inventors surprisingly discovered that the levels of miRNA biomarkers that regulate expression of the proteins in Table 1 are similarly altered by either upregulation or downregulation dependent on the severity of injury or the time since onset of injury. The pattern of miRNA and other biomarkers changes as injury or disease progresses. This may be a result of secondary injury events, delayed cell apoptosis, or other mechanism altering the release of RNA, DNA, or protein. Redell, J, incorporated herein by reference above, illustrates alteration of miRNA biomarkers at 3 hours, and 24 hours. Some miRNAs are upregulated at 3 hours whereas others are only upregulated at 24 hours. Similar results are observed for downregulation of miRNA. As such, the regulation of miRNA biomarkers, the method of their detection, and the temporal alteration in expression of Redell, J, et al., J. Neurosci. Res., 2009; 87:1435-1448 are each incorporated herein by reference as equally applicable to the subject invention. Similarly, the temporal nature of miRNA expression in response to stroke as observed by Jeyaseelan, K, et al., Stroke, 2008; 39:959-966 is also incorporated herein by reference for the particular miRNAs taught therein as well as the methods of isolation, quantification, and detection taught therein.

As such, the invention optionally screens a biological sample for a first and a second biomarker. Greater numbers are similarly operable. GFAP biomarkers are optional first biomarkers. As GFAP is associated with glial cells such as astrocytes, preferably the other biomarker is associated with the health of a different type of cell associated with neural function. More preferably, the other cell type is an axon, neuron, or dendrite. Through the use of an inventive assay inclusive of biomarkers associated with glial cells as well as at least one other type of neural cell, the type of neural cells being stressed or killed as well as quantification of neurological condition results. A synergistic measurement of GFAP biomarker optionally along with at least one additional biomarker and comparing the quantity of GFAP biomarker and the additional biomarker to normal levels of the markers provides a determination of subject neurological condition. Specific biomarker levels that alone or when measured in concert with GFAP biomarker afford superior evaluation of subject neurological condition illustratively include SBDP150 and SBDP145 (calpain mediated acute neural necrosis), SBDP120 (caspase mediated delayed neural apoptosis), UCH-L1 (neuronal cell body damage marker), and MAP-2.

The nature of a particular protein associated with an inventive biomarker allows tight determination of extent, location, and severity of injury. Table 2 represents biological locations of proteins related to inventive biomarkers. It is appreciated that increases in protein, autoantibodies, or RNA, for example, peripherin equates to different abnormalities than increases in autoantibodies or RNA to UCH-L1, SBDPs, MAP-2 and GFAP.

The detection of inventive biomarkers is also operable to screen potential drug candidates or analyze safety of previously identified drug candidates. These assays are optionally either in vitro or in vivo. In vivo screening or assay protocols illustratively include measurement of a biomarker in an animal illustratively including a mouse, rat, or human or other non-human mammals producing a given biomarker in response to neurotoxicity. Studies to determine or monitor levels such as UCH-L1 or SBDP 145 biomarkers are optionally combined with behavioral analyses or motor deficit analyses such as: motor coordination tests illustratively including Rotarod, beam walk test, gait analysis, grid test, hanging test and string test; sedation tests illustratively including those detecting spontaneous locomotor activity in the open-field test; sensitivity tests for allodynia—cold bath tests, hot plate tests at 38° C. and Von Frey tests; sensitivity tests for hyperalgesia—hot plate tests at 52° C. and Randall-Sellito tests; and EMG evaluations such as sensory and motor nerve conduction, Compound Muscle Action Potential (CMAP) and h-wave reflex.

The inventive biomarker analyses are illustratively operable to detect, diagnose, or treat a disease state or screen for chemical or other therapeutics to treat disease or injury. Diseases or conditions illustratively screenable include but are not limited to: myelin involving diseases such as multiple sclerosis, stroke, amyotrophic lateral sclerosis (ALS), chemotherapy, cancer, Parkinson's disease, nerve conduction abnormalities stemming from chemical or physiological abnormalities such as ulnar neuritis and carpel tunnel syndrome, other peripheral neuropathies illustratively including sciatic nerve crush (traumatic neuropathy), streptozotozin (STZ) (diabetic neuropathy), antimitotic-induced neuropathies (chemotherapy-induced neuropathy), experimental autoimmune encephalomyelitis (EAE), delayed-type hypersensitivity (DTH), rheumatoid arthritis, epilepsy, pain, neuropathic pain, and intra-uterine trauma.

To provide correlations between neurological condition and measured quantities of biomarkers of UCH-L1 and others, samples of CSF or serum are collected from subjects with the samples being subjected to measurement of biomarkers. The subjects vary in neurological condition. Detected levels of UCH-L1 biomarkers are optionally then correlated with CT scan results as well as GCS scoring. Based on these results, an inventive assay is developed and validated (Lee et al., Pharmacological Research 23:312-328, 2006). It is appreciated that UCH-L1 biomarkers, in addition to being obtained from CSF and serum, are also readily obtained from blood, plasma, saliva, urine, as well as solid tissue biopsy. While CSF is a preferred sampling fluid owing to direct contact with the nervous system, it is appreciated that other biological fluids have advantages in being sampled for other purposes and therefore allow for inventive determination of neurological condition as part of a battery of tests performed on a single sample such as blood, plasma, serum, saliva or urine.

A biological sample is obtained from a subject by conventional techniques. For example, CSF is obtained by lumbar puncture. In some embodiments, CSF is not obtained by cannulation such as by lumbar puncture or by insertion of a needle, illustratively a butterfly needle, guided transcutaneously into the cistern magna at the time of collection similar to the technique of Nirogi et al., J. Neurosci. Methods, 2009; 178(1):116-119, the contents of which are incorporate herein by reference. Blood is obtained by venipuncture, while plasma and serum are obtained by fractionating whole blood according to known methods. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J. Clin. Microbiol. 38: 3892-3895 (2000).

A biomarker is optionally selective for detecting or diagnosing neurological conditions such as neurotoxic insult and the like. Optionally, a biomarker is both specific and effective for the detection and distinguishing levels of chemical induced neurotoxicity. Such biomarkers are optionally termed neuroactive biomarkers.

It is appreciated that the temporal nature of biomarker presence or activity is operable as an indicator or distinguisher of neurotoxicity. In a non-limiting example, the severity of experimental systemic exposure to MK-801, which causes Olney's lesions, correlates with the temporal maintenance of UCH-L1 in CSF.

Biomarker analyses are optionally performed using biological samples or fluids. Illustrative biological samples operable herein illustratively include, cells, tissues, cerebral spinal fluid (CSF), artificial CSF, whole blood, serum, plasma, cytosolic fluid, urine, feces, stomach fluids, digestive fluids, saliva, nasal or other airway fluid, vaginal fluids, semen, buffered saline, saline, water, or other biological fluid recognized in the art.

In addition to increased cell expression, protein biomarkers optionally also appear in biological fluids in communication with injured cells. Obtaining biological fluids such as cerebrospinal fluid (CSF), blood, plasma, serum, saliva and urine, from a subject is typically much less invasive and traumatizing than obtaining a solid tissue biopsy sample. Thus, samples that are biological fluids are preferred for use in the invention. CSF, in particular, is preferred for detecting nerve damage in a subject as it is in immediate contact with the nervous system and is readily obtainable. Serum as an exemplary biological sample is much easily obtainable and presents much low risk of further injury or side-effect to a donating subject.

After insult, nerve cells in in vitro culture or in situ in a subject express altered levels or activities of one or more proteins or RNA molecules than do such cells not subjected to the insult. Thus, samples that contain nerve cells, e.g., a biopsy of a central nervous system or peripheral nervous system tissue are suitable biological samples for use in the invention. In addition to nerve cells, however, other cells express illustratively αII-spectrin including, for example, erythrocytes, cardiomyocytes, myocytes in skeletal muscles, hepatocytes, kidney cells and cells in testis. A biological sample including such cells or fluid secreted from these cells might also be used in an adaptation of the inventive methods to determine and/or characterize an injury to such non-nerve cells.

A subject as used herein illustratively includes a dog, a cat, a horse, a cow, a pig, a sheep, a goat, a chicken, non-human primate, a rat, guinea pig, hamster, and a mouse. Because the present invention relates to human subjects, a subject for the methods of the invention is optionally a human being.

Subjects who most benefit from the present invention are optionally those suspected of having or at risk for developing abnormal neurological conditions or injury, such as victims of brain injury caused by traumatic insults (e.g., gunshot wounds, automobile accidents, sports accidents, shaken baby syndrome, other percussive injuries), ischemic events (e.g., stroke, cerebral hemorrhage, cardiac arrest), neurodegenerative disorders (such as Alzheimer's, Huntington's, and Parkinson's diseases; prion-related disease; other forms of dementia), epilepsy, substance abuse (e.g., from amphetamines, Ecstasy/MDMA, or ethanol), and peripheral nervous system pathologies such as diabetic neuropathy, chemotherapy-induced neuropathy and neuropathic pain.

To provide correlations between a neurological condition and measured quantities of biomarkers, CSF or serum are optional biological fluids. Illustratively, samples of CSF or serum are collected from subjects with the samples being subjected to measurement of biomarkers. Collection of biological fluids or other biological samples are illustratively prior to or following administering a chemical or biological agent. Illustratively, a subject is optionally administered a chemical agent, such as an agent for drug screening. Prior to administration, at the time of administration, or any desired time thereafter, a biological sample is obtained from the subject. It is preferred that a biological sample is obtained during or shortly after the drug is found in the blood stream of the subject. Illustratively, a biological sample is obtained during the increase in plasma concentration observed following oral dosing. Illustratively, a biological sample is also obtained following peak plasma concentrations are obtained. Optionally, a biological sample is obtained 1, 2, 3, 4, 5, 10, 12, 24 hours or anytime in between after administration. Optionally, a biological sample is obtained 1, 2, 3, 4, 5, 6, 7, days or anytime in between. In some embodiments, a biological sample is obtained 1, 2, 3, 4, weeks or more, or any time in between. It is appreciated that neurotoxicity occurs immediately after administration or is delayed. A biological sample is optionally obtained 1, 2, 3, 6, months or more, or any time in between to detect delayed neurotoxicity. In some embodiments, a subject is continually dosed for hours, days, weeks, months, or years during which time one or more biological samples is obtained for biomarker screening. In some embodiments, phase IV trials are used to monitor the continued safety of a marketed chemical or biological agent. These trials optionally continue for years or indefinitely. As such, any time from prior to administration to years following the first administration, a biological sample is obtained for detection of one or more inventive biomarkers of neurotoxicity.

The subjects vary in neurological condition. Detected levels of one or more biomarkers are optionally then correlated with either recognized or standardized baseline levels or optionally CT scan results as well as GCS scoring. Based on these results, an inventive assay is optionally developed and validated. It is appreciated that neuroactive biomarkers, in addition to being obtained from CSF and serum, are also readily obtained from blood, plasma, saliva, urine, as well as solid tissue biopsy. While CSF is a preferred sampling fluid owing to direct contact with the nervous system, it is appreciated that other biological fluids have advantages in being sampled for other purposes and therefore allow for inventive determination of neurological condition alone or as part of a battery of tests performed on a single sample such as blood, plasma, serum, saliva or urine. Clinical manifestations of neurotoxic insult illustratively include GCS score negative change, seizures, and neurogeneration.

Baseline levels of biomarkers are those levels obtained in the target biological sample in the species of desired subject in the absence of a known neurological condition. These levels need not be expressed in hard concentrations, but may instead be known from parallel control experiments and expressed in terms of fluorescent units, density units, and the like. Typically, in the absence of a neurological condition, one or more SBDPs are present in biological samples at a negligible amount. However, UCH-L1 is a highly abundant protein in neurons. Determining the baseline levels of UCH-L1 or UCH-L1 biomarkers such as mRNA in neurons, plasma, or CSF, for example, of particular species is well within the skill of the art. Similarly, determining the concentration of baseline levels of other biomarkers is well within the skill of the art.

As used herein the term “diagnosing” means recognizing the presence or absence of a neurological or other condition such as neurotoxicity. Diagnosing is optionally referred to as the result of an assay wherein a particular ratio or level of a biomarker is detected or is absent.

As used herein a “ratio” is either a positive ratio wherein the level of the target is greater than the target in a second sample or relative to a known or recognized baseline level of the same target. A negative ratio describes the level of the target as lower than the target in a second sample or relative to a known or recognized baseline level of the same target. A neutral ratio describes no observed change in target biomarker.

As used herein the term “administering” is delivery of a therapeutic to a subject. The therapeutic is a chemical or biological agent administered with the intent to ameliorate one or more symptoms of a condition or treat a condition. As used herein the term “exposing” is used to connote administering to a subject as well as in vitro or in vivo targeted contact with subject cells. A therapeutic is administered by a route determined to be appropriate for a particular subject by one skilled in the art. For example, the therapeutic is administered orally, parenterally (for example, intravenously, by intramuscular injection, by intraperitoneal injection, intratumorally, by inhalation, or transdermally. The exact amount of therapeutic required will vary from subject to subject, depending on the age, weight and general condition of the subject, the severity of the neurological condition that is being treated, the particular therapeutic used, its mode of administration, and the like. An appropriate amount may be determined by one of ordinary skill in the art using only routine experimentation given the teachings herein or by knowledge in the art without undue experimentation.

An exemplary process for detecting the presence or absence of one or more neuroactive biomarkers in a biological sample involves obtaining a biological sample from a subject, such as a human, contacting the biological sample with a compound or an agent capable of detecting of the biomarker being analyzed, illustratively including a primer, a probe, antigen, peptide, chemical agent, or antibody, and analyzing the sample for the presence of the biomarker. It is appreciated that other detection methods are similarly operable illustratively contact with a protein or nucleic acid specific stain.

An inventive process is optionally used to detect UCH-L1 biomarkers and one or more other neuroactive biomarkers in a biological sample in vitro, as well as in vivo. The quantity of expression of UCH-L1 biomarkers in a sample is compared with appropriate controls such as a first sample known to express detectable levels of the marker being analyzed (positive control) and a second sample known to not express detectable levels of the marker being analyzed (a negative control). For example, in vitro techniques for detection of a marker include enzyme linked immunosorbent assays (ELISAs), radioimmuno assay, radio assay, Western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art. Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques.

Any suitable molecules that can specifically bind or otherwise be used to recognize a UCH-L1 biomarker are operative in the invention. A preferred agent for detecting UCH-L1, SBDP 145, GFAP, or other biomarkers such as those listed in Table 1, is an antigen capable of binding to an autoantibody or an antibody capable of binding a biomarker being analyzed. Such antibodies can be polyclonal or monoclonal. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof. Antibodies operable herein are optionally monoclonal or polyclonal.

RNA and DNA binding antibodies are known in the art. Illustratively, an RNA binding antibody is synthesized from a series of antibody fragments from a phage display library. Illustrative examples of the methods used to synthesize RNA binding antibodies are found in Ye, J, et al., PNAS USA, 2008; 105:82-87 the contents of which are incorporated herein by reference as methods of generating RNA binding antibodies. As such, it is within the skill of the art to generate antibodies to RNA based biomarkers.

DNA binding antibodies are similarly well known in the art. Illustrative methods of generating DNA binding antibodies are found in Watts, R A, et al., Immunology, 1990; 69(3): 348-354 the contents of which are incorporated herein by reference as an exemplary method of generating anti-DNA antibodies.

An antibody is optionally labeled. A person of ordinary skill in the art recognizes numerous labels operable herein. Labels and labeling kits are commercially available optionally from Invitrogen Corp, Carlsbad, Calif. Labels illustratively include, fluorescent labels, biotin, peroxidase, radionucleotides, or other label known in the art.

Antibody-based assays are preferred for analyzing a biological sample for the presence of UCH-L1, SBDP 145, GFAP, MAP2, S100b or other biomarkers. Suitable Western blotting methods are known to those of skill in the art. For more rapid analysis (as may be important in emergency medical situations), immunosorbent assays (e.g., ELISA and RIA) and immunoprecipitation assays may be used. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody that specifically binds UCH-L1, SBDP150, SBDP145, MAP2, GFAP, NSE, S100b or one of the other inventive biomarkers under conditions that allow binding of antibody to the biomarker being analyzed. After washing, the presence of the antibody on the substrate indicates that the sample contained the marker being assessed. If the antibody is directly conjugated with a detectable label, such as an enzyme, fluorophore, or radioisotope, the label presence is optionally detected by examining the substrate for the detectable label. Alternatively, a detectably labeled secondary antibody that binds the marker-specific antibody is added to the substrate. The presence of detectable label on the substrate after washing indicates that the sample contained the marker. Alternatively, a sandwich assay is used where a specific primary antibody directed to a biomarker is bound to a solid substrate. A biological sample is incubated with the plate and non-specifically bound material is washed away. A labeled or otherwise detectable secondary antibody is used to bind the biomarker affixed to the substrate by the primary antibody. Detection of secondary antibody binding indicates the presence of the biomarker in the biological sample.

Numerous permutations of these basic immunoassays are also operative in the invention. These include the biomarker-specific antibody, as opposed to the sample being immobilized on a substrate, and the substrate is contacted with UCH-L1, SBDP150, SBDP145, GFAP, MAP2, or another neuroactive biomarker conjugated with a detectable label under conditions that cause binding of antibody to the labeled marker. The substrate is then contacted with a sample under conditions that allow binding of the marker being analyzed to the antibody. A reduction in the amount of detectable label on the substrate after washing indicates that the sample contained the marker.

Although antibodies are one composition operable in the invention because of their extensive characterization, any other suitable agent (e.g., a peptide, an aptamer, or a small organic molecule) that specifically binds a UCH-L1, SBDP150, SBDP 145, GFAP, MAP2, or other biomarker is optionally used in place of the antibody. For example, an aptamer that specifically binds αII spectrin and/or one or more of its SBDPs might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression are known in the art. Agents used in methods for detecting UCH-L1 or another biomarker are conjugated to a detectable label, e.g., an enzyme such as horseradish peroxidase. Agents labeled with horseradish peroxidase can be detected by adding an appropriate substrate that produces a color change in the presence of horseradish peroxidase. Several other detectable labels that may be used are known. Common examples of these include alkaline phosphatase, horseradish peroxidase, fluorescent compounds, luminescent compounds, colloidal gold, magnetic particles, biotin, radioisotopes, and other enzymes. It is appreciated that a primary/secondary antibody system is optionally used to detect one or more biomarkers. A primary antibody that specifically recognizes one or more biomarkers is exposed to a biological sample that may contain the biomarker of interest. A secondary antibody with an appropriate label that recognizes the species or isotype of the primary antibody is then contacted with the sample such that specific detection of the one or more biomarkers in the sample is achieved.

The present invention employs a step of correlating the presence or amount of UCH-L1, SBDP 145, SBDP150, MAP2, GFAP, or other biomarker in a biological sample with the severity and/or type of nerve cell injury. The amount of UCH-L1, as one illustration, in the biological sample is associated with neurotoxic insult created by a neurological condition. The results of an inventive assay to synergistically measure UCH-L1 biomarkers and one or more other biomarkers can help a physician determine the type and severity of injury with implications as to the types of cells that have been compromised. These results are in agreement with CT scan and GCS results, yet are quantitative, obtained more rapidly, and at far lower cost.

The present invention provides a step of comparing the quantity of UCH-L1 biomarker and optionally the amount of at least one other biomarker, illustratively SBDP 145 or SBDP150, to normal levels or one or each to determine the neurological condition of the subject. It is appreciated that selection of additional biomarkers allows one to identify the types of nerve cells implicated in an abnormal neurological condition such as neurotoxicity as well as the nature of cell death in the case of an axonal injury marker, namely an autoantibody directed to a SBDP or GFAP.

The practice of an inventive process provides a test that can help a physician determine suitable therapeutic(s) to administer for optimal benefit of the subject. While the subsequently provided data found in the examples is provided with respect to a full spectrum of brain injury, it is appreciated that these results are applicable to ischemic events, neurodegenerative disorders, prion related disease, epilepsy, chemical or biological agent etiology, and peripheral nervous system pathologies. A gender difference may be present in abnormal subject neurological condition.

An assay for analyzing cell damage or other cellular condition in a subject is also provided. The assay illustratively includes: (a) a substrate for holding a sample isolated from a subject suspected of having a damaged nerve cell, the sample being a fluid in communication with the nervous system of the subject prior to being isolated from the subject; (b) a UCH-L1 biomarker specific binding agent; (c) optionally a binding agent specific for another biomarker such as SBDP 145; and (d) printed instructions for reacting: the UCH-L1 biomarker agent with the biological sample or a portion of the biological sample to detect the presence or amount of UCH-L1 biomarker, and the agent specific for another biomarker with the biological sample or a portion of the biological sample to detect the presence or amount of the at least one other biomarker in the biological sample. The inventive assay can be used to detect neurotoxicity for financial remuneration.

An inventive assay optionally includes a detectable label such as one conjugated to the agent, or one conjugated to a substance that specifically binds to the agent, such as a secondary antibody.

The invention optionally includes one or more therapeutic agents that may alter one or more characteristics of a target biomarker. A therapeutic optionally serves as an agonist or antagonist of a target biomarker or upstream effector of a biomarker. A therapeutic optionally affects a downstream function of a biomarker. For example, Acetylcholine (Ach) plays a role in pathological neuronal excitation and TBI-induced muscarinic cholinergic receptor activation may contribute to excitotoxic processes. As such, biomarkers optionally include levels or activity of Ach or muscarinic receptors. Optionally, an operable biomarker is a molecule, protein, nucleic acid or other that is effected by the activity of muscarinic receptor(s). As such, therapeutics operable in the subject invention illustratively include those that modulate various aspects of muscarinic cholinergic receptor activation.

Specific muscarinic receptors operable as therapeutic targets or modulators of therapeutic targets include the M₁, M₂, M₃, M₄, and M₅ muscarinic receptors.

The suitability of the muscarinic cholinergic receptor pathway in detecting and treating TBI arises from studies that demonstrated elevated ACh in brain cerebrospinal fluid (CSF) following experimental TBI (Gorman et al., 1989; Lyeth et al., 1993a) and ischemia (Kumagae and Matsui, 1991), as well as the injurious nature of high levels of muscarinic cholinergic receptor activation through application of cholinomimetics (Olney et al., 1983; Turski et al., 1983). Furthermore, acute administration of muscarinic antagonists improves behavioral recovery following experimental TBI (Lyeth et al., 1988a; Lyeth et al., 1988b; Lyeth and Hayes, 1992; Lyeth et al., 1993b; Robinson et al., 1990). As such chemical or biological agents that bind to, or alter a characteristic of a muscarinic cholinergic receptor are optionally screened for neurotoxicity of cells or tissues such as during target optimization in pre-clinical drug discovery.

A therapeutic agent, chemical agent, or biological agent, operable in the subject invention is illustratively any molecule, compound, family, extract, solution, drug, pro-drug, or other mechanism that is operable for changing, preferably improving, therapeutic outcome of a subject at risk for or subjected to a neurotoxic insult. An agent is optionally a muscarinic cholinergic receptor modulator such as an agonist or antagonist. An agonist or antagonist may by direct or indirect. An indirect agonist or antagonist is optionally a molecule that breaks down or synthesizes acetylcholine or other muscarinic receptor related molecule illustratively, molecules currently used for the treatment of Alzheimer's disease. Choline mimetic or similar molecules are operable herein. An exemplary list of therapeutics operable herein include: dicyclomine, scoplamine, milameline, N-methyl-4-piperidinylbenzilate NMP, pilocarpine, pirenzepine, acetylcholine, methacholine, carbachol, bethanechol, muscarine, oxotremorine M, oxotremorine, thapsigargin, calcium channel blockers or agonists, nicotine, xanomeline, BuTAC, clozapine, olanzapine, cevimeline, aceclidine, arecoline, tolterodine, rociverine, IQNP, indole alkaloids, himbacine, cyclostellettamines, kainic acid, chloropropionic acid, bromethalin, methotrexate, anti-cancer chemotherapeutics, such as paxlitaxel, and organo-platinum compounds, pentylenetetrazol; antipsychotics, illegal psychoactive drugs, alcohol; derivatives of any of the aforementioned , pro-drugs of any of the aforementioned, and combinations of any of the aforementioned. A therapeutic is optionally a molecule operable to alter the level of or activity of a calpain or caspase. Such molecules and their administration are known in the art.

An inventive method illustratively includes a process for diagnosing a neurological condition in a subject, treating a subject with a neurological condition, or both. In a some embodiments an inventive process illustratively includes obtaining a biological sample from a subject. The biological sample is assayed by mechanisms known in the art for detecting or identifying the presence of one or more biomarkers present in the biological sample. Based on the amount or presence of a target biomarker in a biological sample, a ratio of one or more biomarkers is optionally calculated. The ratio is optionally the level of one or more biomarkers relative to the level of another biomarker in the same or a parallel sample, or the ratio of the quantity of the biomarker to a measured or previously established baseline level of the same biomarker in a subject known to be free of a pathological neurological condition. The ratio allows for the diagnosis of a neurological condition in the subject. An inventive process optionally administers a therapeutic to the subject that will either directly or indirectly alter the ratio of one or more biomarkers.

An inventive process is also provided for detecting, diagnosing or treating a multiple-organ injury. Multiple organs illustratively include subsets of neurological tissue such as brain, spinal cord and the like, or specific regions of the brain such as cortex, hippocampus and the like. Multiple injuries illustratively include apoptotic cell death which is detectable by the presence of biomarkers for caspase induced SBDPs, and oncotic cell death which is detectable by the presence of biomarkers for calpain induced SBDPs. The inventive process illustratively includes assaying for a plurality of biomarkers in a biological sample obtained from a subject wherein the biological is optionally in fluidic contact with an organ subjected to neurotoxic insult or control organ when the biological sample is obtained from the subject. The inventive process determines a first subtype of organ injury in based on a first ratio of a plurality of biomarkers. The inventive process also determines a second subtype of a second organ injury based on a second ration of the plurality of biomarkers in the biological sample. The ratios are illustratively determined by processes described herein or known in the art.

Treatment of a multiple organ injury in the inventive process is illustratively achieved by administering to a subject at least one therapeutic antagonist or agonist effective to modulate the activity of a protein whose activity is altered in response to the first organ injury, and administering at least one therapeutic agonist or antagonist effective to modulate the activity of a protein whose activity is altered in response to a second organ injury.

The subject invention illustratively includes a composition for distinguishing the magnitude of a neurotoxic insult in a subject. An inventive composition is either an agent or a mixture of multiple agents. In an optional embodiment a composition is a mixture. The mixture optionally contains a biological sample derived from a subject. The subject is optionally suspected of having a neurotoxic condition. The biological sample in communication with the nervous system of the subject prior to being isolated from the subject. In inventive composition also contains at least two primary agents, preferably antibodies or primers that specifically and independently bind to at least two biomarkers that may be present in the biological sample. In some optional embodiments the first primary agent is in antibody that specifically binds a ubiquitin carboxyl-terminal hydrolase biomarker, preferably a UCH-L1 biomarker. A second primary agent is preferably an antibody that specifically binds a spectrin breakdown product biomarker such as SBDP 145.

The agents of the inventive composition are optionally mobilized or otherwise in contact with a substrate. The inventive teachings are also optionally labeled with at least one detectable label. In an optional embodiment the detectable label on each agent is unique and independently detectable. Optionally, a secondary agent specific for detecting or binding to the primary agent is labeled with at least one detectable label. In the nonlimiting example the primary agent is a rabbit derived antibody. A secondary agent is optionally an antibody specific for a rabbit derived primary antibody. Mechanisms of detecting antibody binding to an antigen are well known in the art, and a person of ordinary skill in the art readily envisions numerous methods and agents suitable for detecting antigens or biomarkers in a biological sample.

The kit is also provided that encompasses a substrate suitable for associating with the target biomarker in a biological sample. The biological sample is optionally provided with the kit or is obtained by a practitioner for use with an inventive kit. An inventive kit also includes optionally at least two antibodies that specifically and independently bind to at least two biomarkers. The antibodies preferably distinguish between the two biomarkers. Optionally, a first antibody is specific and independent for binding and detecting a first biomarker. A second antibody is specific and independent for binding and detecting a second biomarker. In this way the presence or absence of multiple biomarkers in a single biological sample can be determined or distinguished. In some optional embodiments target biomarkers in the biological sample illustratively include those for biomarkers of αII-spectrin, an αII-spectrin breakdown product (SBDP) such as SBDP 145, a ubiquitin carboxyl-terminal hydrolase, GFAP, and a MAP2 protein. An inventive kit also includes instructions for reacting the antibodies with the biological sample or a portion of the biological sample so as to detect the presence of or amount of the biomarkers in the biological sample.

In the kit, the biological sample can be CSF or blood, and the agent is optionally an antibody, aptamer, primer, probe, or other molecule that specifically binds at least one biomarker for a neurological condition. Suitable agents are described above. The kit can also include a detectable label such as one conjugated to the agent, or one conjugated to a substance that specifically binds to the agent (e.g., a secondary antibody).

The invention employs a step of correlating the presence or amount of a biomarker in a biological sample with the severity and/or type of nerve cell (or other biomarker-expressing cell) toxicity. The amount of biomarker(s) in the biological sample directly relates to severity of nerve tissue toxicity as a more severe injury damages a greater number of nerve cells which in turn causes a larger amount of biomarker(s) to accumulate in the biological sample (e.g., CSF; serum). Whether a neurotoxic insult triggers an apoptotic and/or necrotic type of cell death can also be determined by examining the biomarkers for SBDPs such as SBDP 145 present in the biological sample. Necrotic cell death preferentially activates calpain, whereas apoptotic cell death preferentially activates caspase-3. Because calpain and caspase-3 SBDPs can be distinguished, measurement of these markers indicates the type of cell damage in the subject. For example, necrosis-induced calpain activation results in the production of SBDP150 and SBDP145; apoptosis-induced caspase-3 activation results in the production of SBDP150i and SBDP120; and activation of both pathways results in the production of all four markers. Also, the level of or kinetic extent of UCH-L1 biomarkers present in a biological sample may optionally distinguish mild injury from a more severe injury. In an illustrative example, severe MCAO (2 h) produces increased UCH-L1 in both CSF and serum relative to mild challenge (30 min) while both produce UCH-L1 levels in excess of uninjured subjects. Moreover, the persistence or kinetic extent of the markers in a biological sample is indicative of the severity of the neurotoxicity with greater toxicity indicating increases persistence of UCH-L1 or SBDP biomarkers in the subject that is measured by an inventive process in biological samples taken at several time points following injury.

The results of such a test can help a physician determine whether the administration a particular therapeutic such as calpain and/or caspase inhibitors or muscarinic cholinergic receptor antagonists might be of benefit to a patient. This application may be especially important in detecting age and gender difference in cell death mechanism.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of rat tissue, a person having ordinary skill in the art recognizes that similar techniques and other techniques know in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained.

### Example 1

Materials for Biomarker Analyses. Sodium bicarbonate, (Sigma Cat #: C-3041), blocking buffer (Startingblock T20-TBS) (Pierce Cat#: 37543), Tris buffered saline with Tween 20 (TBST; Sigma Cat #: T-9039). Phosphate buffered saline (PBS; Sigma Cat #: P-3813); Tween 20 (Sigma Cat #: P5927); Ultra TMB ELISA (Pierce Cat #: 34028); and Nunc maxisorp ELISA plates (Fisher). Monoclonal and polyclonal UCH-L1 antibodies are made in-house or are obtained from Santa Cruz Biotechnology, Santa Cruz, Calif. Antibodies directed to αII-spectrin and breakdown products (SBDP) as well as to MAP2 are available from Santa Cruz Biotechnology, Santa Cruz, Calif. Labels for antibodies of numerous subtypes are available from Invitrogen, Corp., Carlsbad, Calif. Protein concentrations in biological samples are determined using bicinchoninic acid microprotein assays (Pierce Inc., Rockford, Ill., USA) with albumin standards. All other necessary reagents and materials are known to those of skill in the art and are readily ascertainable.

### Example 2

### In vitro Drug Candidate Screening for Neurotoxicity

Mouse, rat cortical or hippocampal primary neurons are cultured for 21 DIV, and the dose dependent responses of drugs are investigated. Cultured cells are exposed to various concentrations of: Glutamate (0.01 to 1000 μM) in 10 μM glycine both in HBSS; B) 0.01 to 100 μM Kainate in culture media; C) H₂O₂ (0.001 to 1000 μM) in culture media; C) Zinc (0.01 to 1000 μM) in culture media; D) U0126 (0.001 to 100 μM) in culture media; and E) and equal volume of culture media as a control. Glutamate treatment is performed for 30 minutes after which the cells are washed and the HBSS is replaced with culture media and analyzed. The remaining candidates are treated for 24 hours and analyzed. The levels of intracellular UCH-L1 and SBDP 145 are analyzed following cell lysis and screening of the lysates by ELISA using anti-UCH-L1 and SBDP 145 specific antibodies. The levels of UCH-L1 are increased following exposure particularly to Glutamate and H₂O₂.

### Example 3

### Screening for Neurotoxicity of Developmental Neurotoxicant Compounds

ReNcell CX cells are obtained from Millipore (Temecula, Calif.). Cells frozen at passage 3 are thawed and expanded on laminin-coated T75 cm² tissue culture flasks (Corning, Inc., Corning, N.Y.) in ReNcell NSC Maintenance Medium (Millipore) supplemented with epidermal growth factor (EGF) (20 ng/ml; Millipore) and basic fibroblast growth factor (FGF-2) (20 ng/ml; Millipore). Three to four days after plating (e.g., prior to reaching 80% confluency), cells are passaged by detaching with accutase (Millipore), centrifuging at 300×g for 5 min and resuspending the cell pellet in fresh maintenance media containing EGF and FGF-2. For all experiments, cells are replated in laminin-coated costar 96-well plates (Corning, Inc., Corning, N.Y.) at a density of 10,000 cells per well.

Immunocytochemical experiments are conducted to determine the level of UCH-L1 and SBDP 145 in cells prior to and following 24 hours of exposure to 1 nM-100 μM of methyl mercury chloride, trans-retinoic acid, D-amphetamine sulfate, cadmium chloride, dexamethasone, lead acetate, 5,5-diphenylhydantoin, and valproic acid essentially as described in Breier J M et al, Toxicological Sciences, 2008; 105(1):119-133, the contents of which are incorporated herein by reference. Cells are fixed with a 4% paraformaldehyde solution and permeabilized in blocking solution (5% normal goat serum, 0.3% Triton X-100 in phosphate-buffered saline). Fluorescein labeled anti-UCH-L1 Antibody #3524 (Cell Signaling Technology, Danvers, Mass.) is incubated with the fixed cells overnight at 4° C. overnight and visualized using a Nikon TE200 inverted fluorescence microscope with a 20× objective. Images are captured using an RT Slider camera (Model 2.3.1., Diagnostic Instruments, Inc., Sterling Heights, Mich.) and SPOT Advantage software (Version 4.0.9, Diagnostic Instruments, Inc.).

### Example 4

### Acute Oral In vivo Drug Candidate Screening for Neurotoxicity

Female Sprague-Dawley rats (Charles River Laboratories, Inc., Wilmington, Mass.) are dosed with methamphetamine (40 mg/kg as four 10 mg/kg intraperitoneal injections (i.p.) In 1 h interval) (n=8) or cancer drug cisplatin 10 mg/kg (single i.p. injection) (n=4). Anesthesia is performed with intraperitoneal injections of pentobarbital (50 mg/kg). The test substance can also be administered in a single dose by gavage using a stomach tube or a suitable intubation cannula. Animals are fasted prior to dosing. A total of four to eight animals of are used for each dose level investigated.

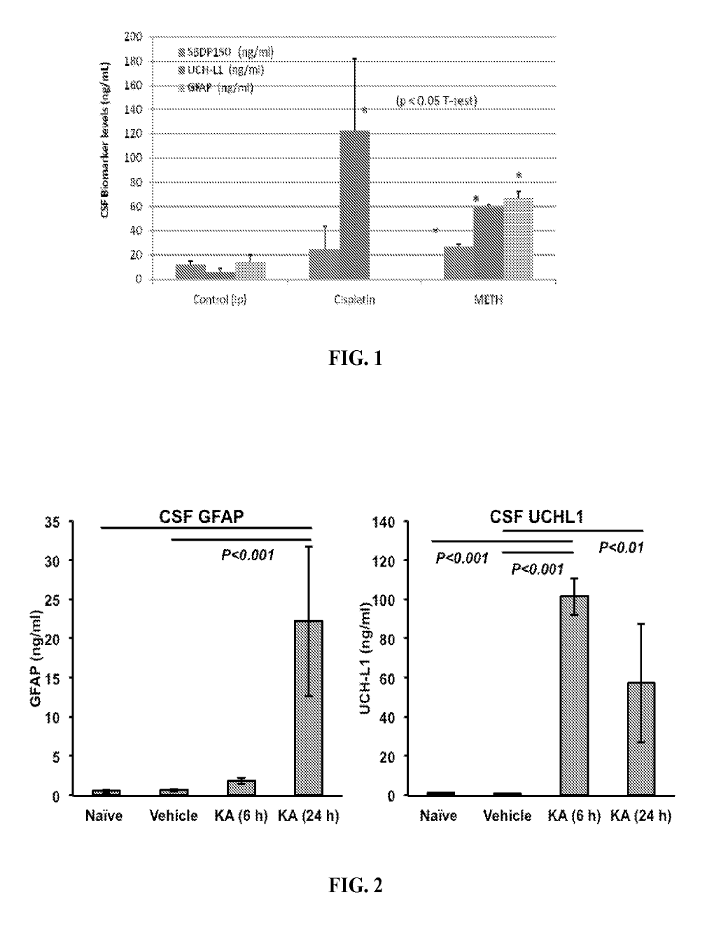

30, 60, 90, and 120 minutes following dosing, the rats are sacrificed by decapitation and blood is obtained by cardiac puncture. The levels of biofluid UCH-L1 and SBDP 150 and GFAP are analyzed by sandwich ELISA or Western blot by using UCH-L1 and SBDP 150 and GFAP specific antibodies. Relative to control animals, neurotoxic levels of methamphetamine induce increased CSF concentrations of both UCH-L1 and SBDP 150 and GFAP. Cisplatin increased UCH-L1 and SBDP150 levels, as shown in FIG. 1.

### Example 5

### Increased Levels of Brain Injury Biomarkers: UCHL 1, GFAP and αll-Spectrin Break Down Products in Rat Model of Kainic Acid-Mediated Neurotoxicity

Male Sprague-Dawley rats (Harlan: Indianapolis, Ind.) with weight from 180 to 200 g are used. The rats are allowed free access to a normal laboratory diet and chlorinated potable water. All rats are acclimated for at least one week to the housing facilities and diet before being used in the study. The controls in the animal room are set to maintain a temperature of 20° C. to 24° C. and 30% to 70% relative humidity. Rats are maintained on a twelve-hour light/dark cycle.

Animals received single subcutaneous injections of Kaininc acid (Sigma, Chemical, St. Louis, Mo., USA) at dose 9 mg/kg and sacrificed at 24 h after injection. Separate control/treatment groups of animals are used. Brain tissues and CSF sample are collected at 6, 24, 48 and 72 h time points after injection.

At the appropriate time points, animals are anesthetized with 4% isoflurane in oxygen as a carrier gas for 4 minutes, followed by maintenance anesthesia of 2-3% isoflurane in the same carrier gas. The rats are placed in a stereotaxic instrument and about 100 μl of CSF from the cisterna magna of each animal is collected transcutaneously through 25 gauge needle attached to polyethylene tubing. CSF samples are frozen on dry ice immediately after collection. After CSF collection animals are removed from the stereotaxic instrument and anesthesia nose cone and the animal, still under anesthesia, is immediately sacrificed by decapitation. Cortex, hippocampus, cerebellum and striatum will be rapidly dissected out, rinsed in cold PBS and snap frozen in liquid nitrogen. To obtain brain tissue for IHC animals are euthanized with lethal dose of pentobarbital, transcardically perfused with 4% paraformaldehyde and whole brains are removed, processed and embedded in paraffin.

The levels and cellular localization TBI biomarkers are examined using ELISA, Western blot and immunohistochemical (IHC) analysis on paraffin-embedded 6 μm brain sections.

Immunohistochemical Analysis

IHC is performed on paraffin-embedded 6 μm brain sections. Slides are deparaffinized, incubated for 10 min at 95° C. in Trilogy solution (Cell Marque, Hot Springs, Ak.) for antigen retrieval, blocked for endogenous peroxides and incubated with 1 Abs (GFAP, SBDP145, SBDP150, SBDP120 and Casp 3) overnight at 4° C. followed by treatments with secondary Abs (LSAB+, # K0679, Dako). The staining is visualized with 3,3′-diaminobenzidine (DAB) (Dako, Carpinteria, Calif.) for brown color development. Sections are counterstained with Hematoxylin (Dako, Carpinteria, Calif.). Negative controls are performed by treatment only with species-matched secondary antibodies.

Immunoblotting Analysis

After SDS-gel electrophoresis in Tris-glycine buffer system and electrotransfer, blotting membranes are blocked for 1 hour at ambient temperature in 5% non-fat milk in Tris-buffered saline (TBS) and then in TBS containing 0.05% Tween-2 (TBST), then incubated in primary monoclonal anti-αII-spectrin antibodies (Biomol, Plymouth Meeting, Pa., USA) diluted ˜1:3000 (3.5 ul/10 ml) in PBS as recommended by the manufacturer at 4° C. overnight. This is followed by three washes with TBST and a 2 hour incubation at ambient temperature with a secondary antibody linked to biotinylated secondary antibody (Amersham, Cat # RPN1177v1) followed by a 30 min incubation with strepavidin-conjugated alkaline phosphatase (colorimetric method). Then a colorimetric development is performed with a one-step BCIP/NBT reagent (KPL, Cat #50-81-08). Molecular weight of intact αll-spectrin protein and αll-spectrin breakdown products (SBDPs) are assessed by running alongside rainbow colored molecular weight standards (Amersham, Cat # RPN800V). Semi-quantitative evaluation of levels of αll-spectrin and of its breakdown products SBDP150 and SBDP145 and SBDP120 is performed using computer-assisted high-resolution flatbed scanner Epson XL3500 and densitometric image analysis with Image J software (NIH). Uneven loading of samples onto different lands might occur despite careful protein concentration determination and careful sample handling and gel loading (20 mg per land). To overcome this source of variability, Western blot is performed using the same sample against β-actin (monoclonal, Sigma, # A5441) as a control.

UCH-L1 Biomarker Sandwich ELISA Analysis Method

Serum and CSF sample concentrations of UCH-L1 are measured using a UCH-L1 sandwich enzyme-linked immunosorbent assay (ELISA) version lb modified from a protocol previously reported (Papa L. et al., 2010; Liu M. et al., 2009). Both mouse monoclonal antibody (capture antibody) and rabbit polyclonal antibody (detection antibody) are made in-house against recombinant human UCH-L1 full length protein and partial protein respectively). Both are affinity purified using target-protein-based affinity column. Their specificity to only target protein (UCH-L1) is confirmed by immunoblotting. Reaction wells are coated with capture antibody (5 μg/mL purified mouse monoclonal anti-human UCHL1) in 0.05 M sodium bicarbonate, pH 9.6 and incubated overnight at 4° C. Plates are then washed with 350 μL/well blocking buffer (Tris buffer saline with 0.02% Tween-20 (v/v); TBST]) and incubated further with 300 μL/well TBST for 30 min at ambient temperature with gentle shaking Antigen standard (UCH-L1 standard curve: 0, 0.06-15 ng/mL, unknown samples (1-10 μL CSF or 20 μL of serum) or assay internal control samples are incubated with detection antibody (rabbit polyclonal antihuman UCH-L1, made in-house; 0.72 μg/mL; 100 μL total vol.) overnight. Afterward the capture antibody coated plate is incubated with detection antibody-sample mixture for 1.5 h at room temperature, it is washed using an automatic plate washer (each well rinsed with 350 μL with wash buffer [TBST]). Secondary anti-rabbit-IgG HRP (Amersham Biosciences; 1/2000 dilution) in blocking buffer is then added to wells (100 μL) at 100 μL/well, and the plates are further incubated at room temperature for 1 h. Finally, the wells are developed with substrate solution: Ultra-TMB ELISA 100 μL/well (Pierce #34028) with incubation for 10 min, plate read at 450 nm with a 96-well spectrophotometer (Molecular Device Spectramax 190).

GFAP Biomarker Sandwich ELISA Method

Serum and CSF sample concentrations of GFAP are measured using a GFAP sandwich enzyme-linked immunosorbent assay (ELISA) version 2a. Both mouse monoclonal antibody (capture antibody) and rabbit polyclonal antibody (detection antibody) are made in-house against recombinant human GFAP full length protein). They are Protein A affinity purified, respectively. Their specificity to only target protein (GFAP) is confirmed by immunoblotting with purified human GFAP (not shown). Reaction wells are coated with capture antibody (5 μg/mL, 100 μL/well purified mouse monoclonal anti-human GFAP) in 0.05 M sodium bicarbonate, pH 9 and incubated 8 h to overnight at 4° C. Plates are then washed with 350 μL/well blocking buffer (Tris buffer saline with 0.02% Tween-20 (v/v); TBST]) and incubated further with 300 μL/well TBST for 30 min at ambient temperature with gentle shaking Antigen standard (GFAP standard curve: 0.02-20 ng/well, unknown samples (3-10 μL CSF or 10-30 μL of serum) or assay internal control samples are incubated with the detection coated plate for 2 h at room temperature. Afterward the plate is washed using an automatic plate washer (each well rinsed with 350 μL with wash buffer [TBST]). This is followed by incubation with detection antibody (rabbit polyclonal anti-human GFAP, 0.25 μg/mL) for 1.5 hrs at room temperature. After further washing, secondary anti-rabbit-IgG HRP (Jacksonville Immuno Research Lab; 1/4000) in blocking buffer is then added to wells at 100 μL/well, and the plates are further incubated at room temperature for 1 h. Finally, the wells are developed with substrate solution: Ultra-TMB ELISA 100 μL/well (Pierce #34028) with incubation for 5-10 min, and plate is read at 450 nm with a 96-well spectrophotometer (Molecular Device Spectramax 190). The Interassay CV=2.1% to 13.0%, while interassay CV=1.0% to 10.0% within the assay dynamic range. Limit of detection (LOD) is determined to be 0.020 ng/mL. For samples with undetectable (ND) levels, they are assigned 50% of the LOD (i.e. 0.010 ng/mL). If sample yields a signal over the quantification range, samples will be diluted and reassayed. As negative controls, we noted that if anti-GFAP capture or detection antibodies are substituted with non-immune normal IgG (mouse) or (rabbit) respectively, no target signals are detected.

SBDP145 Biomarker Sandwich ELISA Method

The SBDP145 ELISA utilizes a proprietary rabbit polyclonal antibody for solid phase immobilization and a proprietary mouse monoclonal antibody conjugated to HRP for detection. The test sample is allowed to react sequentially with these antibodies, resulting in SBDP145molecules being sandwiched between the two antibodies. Detection includes a biotinyl-tyramide amplification step, and is based on a chemiluminescent substrate. Quantitative determination of the biomarker concentration is achieved by comparing the unknown sample result to a standard curve obtained from the same assay. Target concentrations are reported in ng/ml. This assumes that the spectrin breakdown product detected in the sample has a similar MW as that of the calibrator, i.e.145 kDa. If the actual breakdown product has a different or unknown MW, then the reported values should be considered to be relative concentrations only.

SBDP120 Biomarker Sandwich ELISA Method

The SBDP-120 ELISA utilizes a proprietary rabbit polyclonal antibody for solid phase immobilization, and a proprietary mouse monoclonal antibody conjugated to HRP for detection. The test sample is allowed to react sequentially with these antibodies, resulting in SBDP120 molecules being sandwiched between the two antibodies. Detection includes a biotinyl-tyramide amplification step, and is based on a colorimetric (TMB) substrate. Quantitative determination of the biomarker concentration is achieved by comparing the unknown sample signal intensities to a standard curve, obtained from the calibrators run in the same assay. Target concentrations are reported in ng/ml. This assumes that the spectrin breakdown product detected in the sample has a similar MW as that of the calibrator, i.e. 120 kDa. If the actual breakdown product has a different or unknown MW, then the reported values should be considered to be relative concentrations only.

SBDP150 Biomarker Sandwich ELISA Method

The SBDP150 ELISA utilizes a proprietary goat polyclonal antibody for solid phase immobilization, and a proprietary mouse monoclonal antibody conjugated to HRP for detection. The test sample is allowed to react sequentially with these antibodies, resulting in SBDP150 molecules being sandwiched between the two antibodies. Detection includes a biotinyl-tyramide amplification step, and is based on a colorimetric (TMB) substrate. Quantitative determination of the biomarker concentration is achieved by comparing the unknown sample signal intensities to a standard curve, obtained from the calibrators run in the same assay. Target concentrations are reported in ng/ml. This assumes that the spectrin breakdown product detected in the sample has a similar MW as that of the calibrator, i.e. 150 kDa. If the actual breakdown product has a different or unknown MW, then the reported values should be considered to be relative concentrations only.

Results

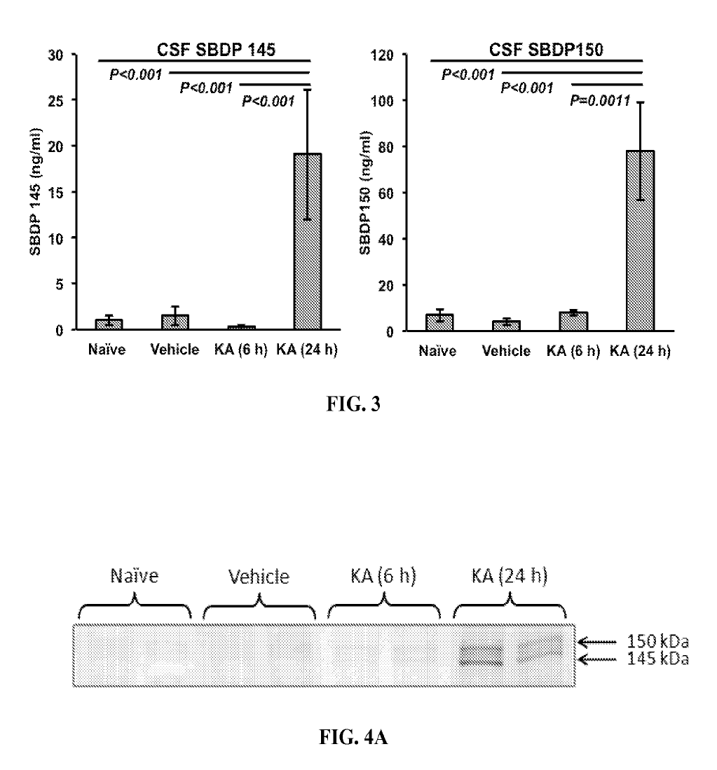

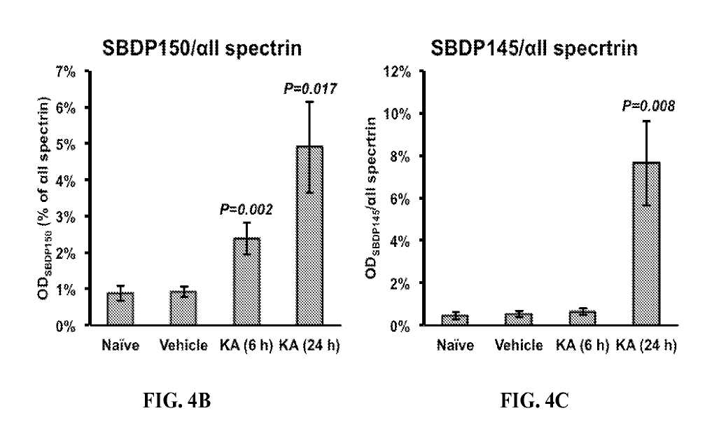

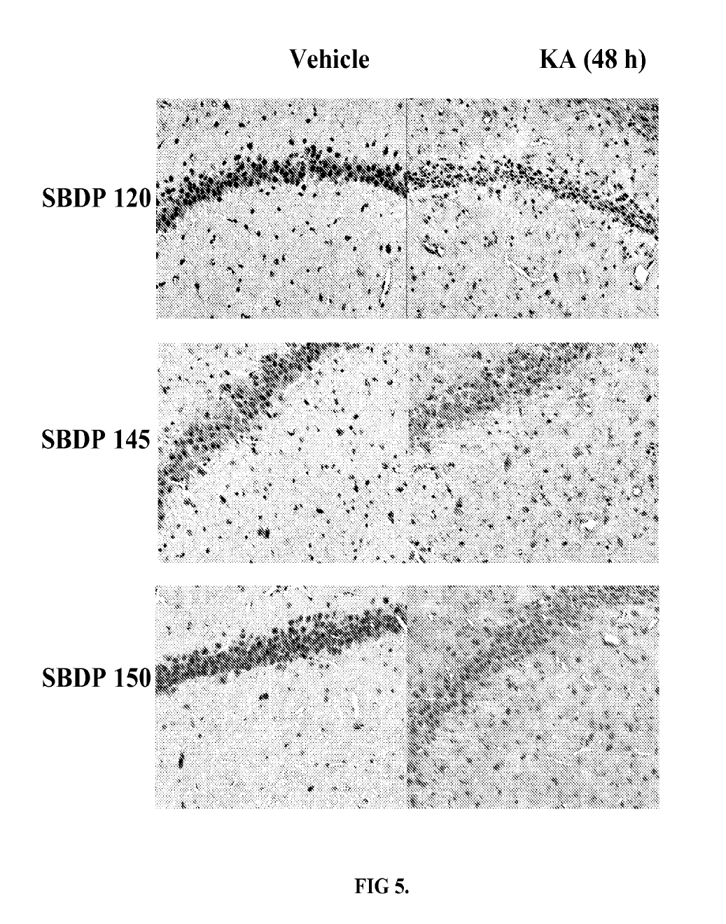

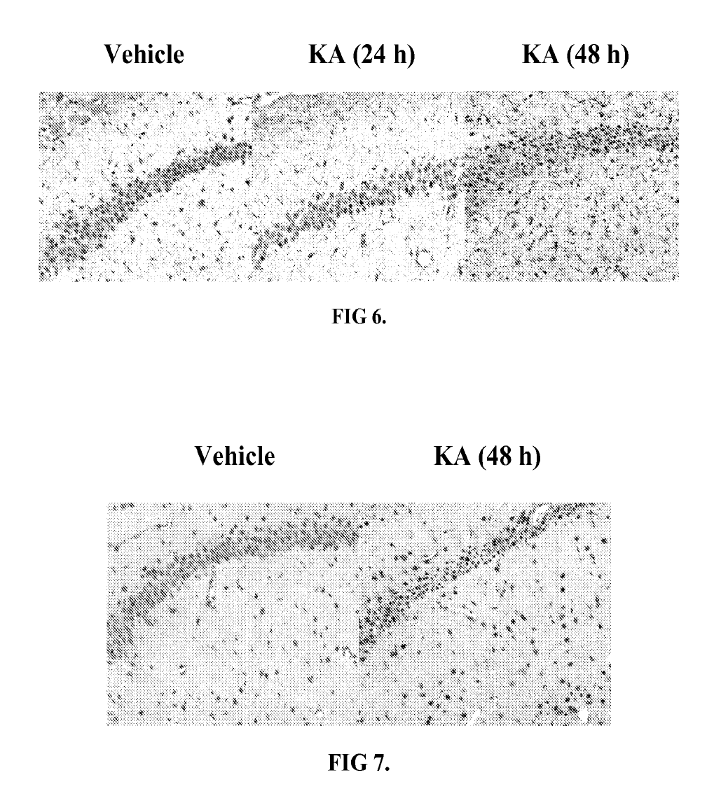

Experiments are performed in two groups of animals: control (saline injection) and treatment (KA injection). Western Blot analysis indicated the decrease in intact αll-spectrin level and appearance of is breakdown fragments produced by both calpain (SBDP150 and SBDP145) and by caspase-3 (SBDP120) in hippocampus and to a lesser extent in cortex in KA group. IHC analysis showed increased level of GFAP, SBDP150, SBDP145 and SBDP120 in hippocampus and cortex of the animal that have undergone KA injection, but not in the animals from control group. ELISA analysis indicated the increase in expression of UCHL 1, GFAP and buildup of αll-spectrin break down products (SBDP150, SBDP145 and SBDP120) in CSF of the KA groups, as compared with control group with the results being shown in FIGS. 2-7.

### Example 6

### Increased Levels of Brain Injury Biomarkers: UCHL 1, GFAP and Neurofilament Protein (NF)-H Isolated from Blood Plasma in Rat Model of Kainic Acid-Mediated Neurotoxicity

The process of Example 5 is repeated with blood plasma as the subject sample source the rats in lieu of CSF. Biomarker levels are observed to be elevated and still correlate with kainic acid exposure as a function of time similar to that shown in FIGS. 2-7.

### Example 7

### Increased Levels of Brain Injury Biomarkers: UCHL 1, GFAP and Neurofilament Protein (NF)-H Isolated from CSF in Rat Model of Kainic Acid-Mediated Neurotoxicity

The process of Example 5 is repeated with rats exposed to single and repeated intraperitoneal injections of paclitaxel at doses 16 mg/kg and 32 mg/kg to induce selective dysfunction of high-diameter myelinated fibers and causing nociceptive peripheral neuropathy in the rats.

Cisplatin is also administered to healthy rats in single intraperitoneal dose of 2 or 10 mg/kg to cause the accumulation of Pt-DNA adducts within 24 hr and these correlated with the severity of peripheral neuropathy. Neuronal degeneration is detected by either silver staining (CNS) or toluidine blue (PNS-tibia or sciatic nerves) and quantified. Biomarker levels are observed to be elevated and correlate with paclitaxel or cisplatin exposure as a function of time similar to that shown in FIGS. 2-7.

### Example 8

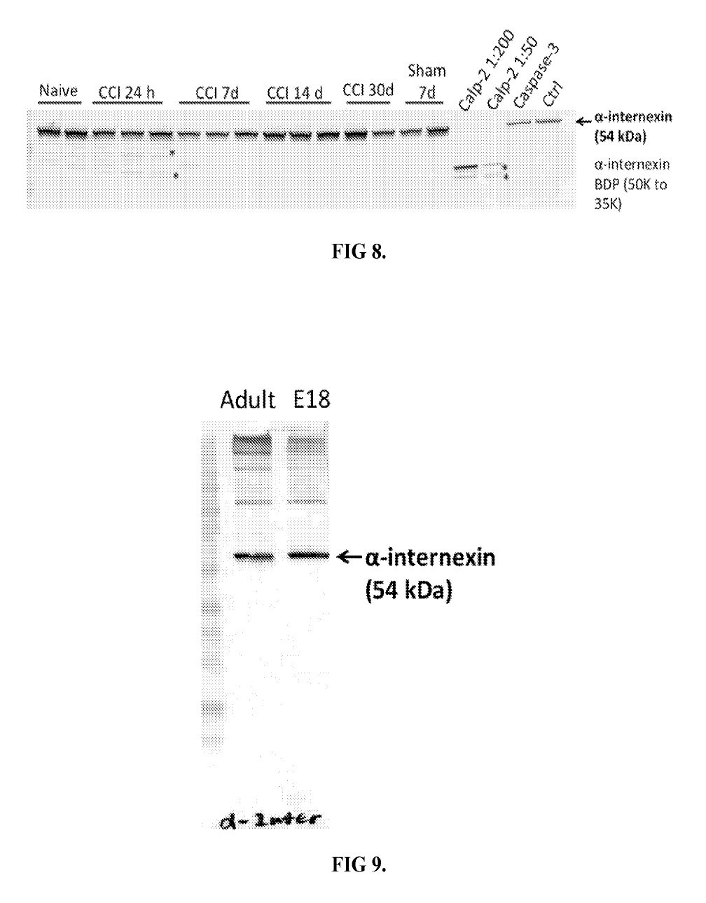

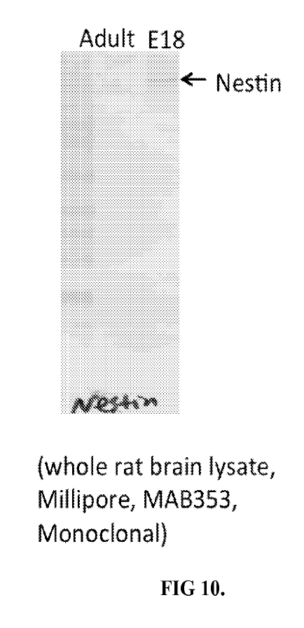

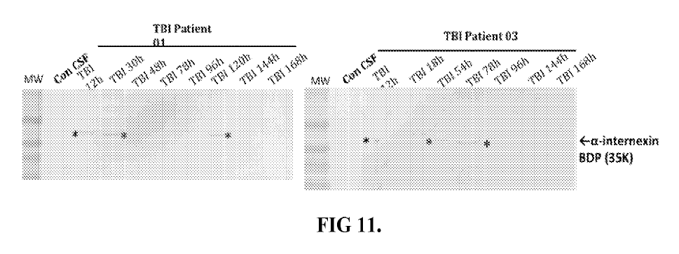

Experiments for alpha-internexin and nestin are performed which proved that nestin and alpha-internexin and breakdown products thereof are exemplary biomarkers for neuronal injury, including TBI, and neurotoxicity. In control CSF, no α-internexin nor α-internexin-BDP are detected by immunblots as shown in FIG. 8. In contrast, with serial CSF samples from two severe TBI patients (01 and 03), a 35 kDa α-internexin-BDP (*) is detected in several acute time points (within 12-30 h) and delayed time points (78-144 h). 1° Antibody: anti-α-internexin (dilution: 1:500); 2° Antibody: Goat-Anti-mouse IgG AP Conjugate (Dilution: 1:5,000). FIG. 9 shows protein from whole rat brain lysate from adult or embryonic day 18 Sprague-Dawley rats is isolated and immunoblotted for alpha-internexin. Thus, α-internexin is an exemplary neurofilament damage marker after adult and pediatric brain injury, as well as after exposure to kainic acid according to the protocol of Example 5. Tissue probed: whole rat brain lysate, primary antibody: EnCor, MCA-2E3, Monoclonal. FIG. 10 shows protein from whole rat brain lysate isolated from adult or embryonic day 18 Sprague-Dawley rats is immunoblotted for nestin, a developmentally regulated protein. Thus, nestin is an exemplary neuro-progenitor cell marker. (tissue probed: whole rat brain lysate, primary antibody: anti-nestin, Millipore, MAB353, Monoclonal). Finally, FIG. 11 illustrates in control CSF, no α-internexin nor α-internexin-BDP is detected by immunoblots. In contrast, with serial CSF samples from two severe TBI patients (01 and 03), a 35 kDa α-internexin-BDP (*) is detected in several acute time points (within 12-30 h) and delayed time points (78-144 h). 1° Antibody: anti-α-internexin (dilution: 1:500); 2° Antibody: Goat-Anti-mouse IgG AP Conjugate (Dilution: 1:5,000).

Patents and publications mentioned in the specification are indicative of the levels of those skilled in the art to which the invention pertains. These patents and publications are incorporated herein by reference to the same extent as if each individual application or publication is specifically and individually expressed explicitly in detail herein.

The foregoing description is illustrative of particular embodiments of the invention, but is not meant to be a limitation upon the practice thereof. The following claims, including all equivalents thereof, are intended to define the scope of the invention.

All references mentioned herein are each incorporated herein by reference as if the contents of each reference are fully and explicitly included for the materials for which each reference is cited.

1. A process for screening neurotoxic insult comprising:
optionally exposing a cell to a chemical or biological agent suspected to be a neurotoxin;
assaying a biological sample of a subject for the presence of one or more biomarkers of a neurotoxicity; and
detecting the neurotoxic insult based on the presence of said one or more of said biomarkers in said sample.

2. The process of claim 1 wherein said assaying is for the presence of two biomarkers of a neurological condition wherein said detecting is based on a ratio of said two biomarkers in said sample.

3. The process of claim 1 wherein said biomarker is a protein selected from the group consisting of:
a ubiquitin carboxyl-terminal hydrolase-L1 (UCH-L1); spectrin; a spectrin breakdown product (SBDP); MAP1, MAP2; GFAP, ubiquitin carboxyl-terminal esterase; ubiquitin carboxyl-terminal hydrolase; a neuronally-localized intracellular protein; MAP-tau; C-tau; Poly (ADP-ribose) polymerase (PARP); a collapsin response mediator protein, synaptotagmin, βIII-tubulin, S100β; neuron-specific enolase, neurofilament protein light chain, nestin, α-internexin; breakdown products thereof, post-translationally modified forms thereof, derivatives thereof, and combinations thereof.

4. The process of claim 1 wherein said biomarker is at least one of a ubiquitin carboxyl-terminal hydrolase, SBDP150, SBDP145, SBDP150i, SBDP120, MAP1, MAP2,GFAP, synaptotagmin, βIII-tubulin, or S100β.

5. The process of claim 2 wherein said biomarker ratio is greater than 2.

6. The process of claim 2 wherein said biomarker ratio is less than 0.5.

7. The process of claims 1 wherein said biomarker is a RNA biomarker.

8. The process of claim 7 wherein said RNA biomarker is a miRNA.

9. The process of claim 1 wherein said biomarker is an autoantibody directed toward a protein selected from the group consisting of:
a ubiquitin carboxyl-terminal hydrolase-L1; GFAP; spectrin; a spectrin breakdown product (SBDP); Nestin; alpha-internexin; MAP1, MAP2; ubiquitin carboxyl-terminal esterase; a neuronally-localized intracellular protein; MAP-tau; C-tau; Poly (ADP-ribose) polymerase (PARP); a collapsin response mediator protein (CRMP); breakdown products thereof, post-translationally modified forms thereof, derivatives thereof, and combinations thereof.

10. The process of claim 9 wherein said biomarker is an autoantibody to at least one of: ubiquitin carboxyl-terminal hydrolase-L1, SBDP150, SBDP145, SBDP150i, SBDP120, MAP1, MAP2 or GFAP, synaptotagmin, βIII-tubulin, or S100β.

11. The process of claim 1 wherein said biological sample is selected from the group consisting of: whole blood, plasma, serum, CSF, urine, saliva, sweat, tears, isolated cells, cell lysate, cell releasate, tissue, tissue lysate, and tissue releasate.

12. The process of claim 1 wherein the step of exposing the cell to said chemical or biological agent is present.

13. The process of claim 1 wherein the step of exposing the cell to said chemical or biological agent is present and said biological agent is at least one of kainic acid, chloropropionic acid, bromethalin, methotrexate, anti-cancer chemotherapeutics, or pentylenetetrazole (PTZ).

14. The process of claim 13 wherein the biological agent is kainic acid.

15. The process of claim 14 wherein the neurotoxic insult has a clinical manifestation of kainate-induced seizures.

16. The process of claim 13 wherein the neurotoxic insult has a clinical manifestation of seizures.

17. The process of claim 13 wherein the neurotoxic insult has a clinical manifestation of neurodegeneration.

18. The process of claim 17 wherein the neurodegeneration is caused by Alzheimer's disease.

19. The process of claim 13 wherein said chemotherapeutic is paxlitaxel or an organo-platinum compound.

20. The process of claim 13 further comprising reducing a quantity of said chemical or biological agent; and assaying a second biological sample from the subject for the presence of said chemical or biological agent to determine an amount below which the neurotoxic insult is not observed.
